(19) United States
(12) Patent Application Publication
(10) Pub. No.: US 2024/0180465 A1
(43) Pub. Date: Jun. 6, 2024

(54) MICRONEEDLE STRUCTURE AND METHOD FOR PRODUCING SAME

(71) Applicant: LINTEC CORPORATION, Tokyo (JP)

(72) Inventor: Yosuke KOMA, Tokyo (JP)

(21) Appl. No.: 18/553,432

(22) PCT Filed: Mar. 31, 2022
(86) PCT No.: PCT/JP2022/016676

§ 371 Date: Sep. 29, 2023

(30) Foreign Application Priority Data
Mar. 31, 2021 (JP) 2021-062453

(51) Int. Cl. A61B 5/15; A61B 5/145; A61L 31/06; A61L 31/14

(52) U.S. Cl. CPC A61B 5/150984; A61B 5/150022; A61L 31/06; A61L 31/148; A61B 5/14532; A61B 2562/12

(57) ABSTRACT

Microneedle structure of the present invention includes a needle-shaped portion on one surface side of a base material. The base material has fluid permeability in its thickness direction. The needle-shaped portion is composed of a composition that contains a low-melting-point resin having a melting point of 150° C. or lower. The needle-shaped portion has a surface and an interior that are formed with hole portions. The method for producing a microneedle structure of the present invention includes a bonding step of heating a composition containing a low-melting-point resin having a melting point of 150° C. or lower to bond the heated low-melting-point resin and the base material. Thus, the microneedle structure and the method for producing the microneedle structure are provided, in which the influence of high temperature on the base material is reduced and the degree of freedom in selecting the base material is high.

### TECHNICAL FIELD

The present invention relates to a microneedle structure and a method for producing the same.

### BACKGROUND ART

In recent years, as a means for transdermal delivery of active substances having pharmaceutical, medical, or cosmetic efficacy, microneedles that are less burdensome for bodies have been applied as substitute for injection needles. For example, Patent Document 1 discloses a microneedle that includes a microneedle-shaped biocompatible matrix and porous particles provided on or at least partially in the biocompatible matrix. In Patent Document 1, projecting portions are not provided with holes, and porous particles containing a drug or the like are provided inside the projecting portions or on their surfaces. When the projecting portions of the microneedle are pierced into the skin, the drug is released from the porous particles of the projecting portions thereby to achieve transdermal delivery.

### PRIOR ART DOCUMENTS

### Patent Documents

[Patent Document 1] JP2016-78474A

### SUMMARY OF THE INVENTION

### Problems to be Solved by the Invention

When it is desired to pierce the patient's skin with a microneedle to collect body fluid such as interstitial fluid and apply the microneedle to an analysis patch for analyzing the body fluid in order to more appropriately understand the patient's medical condition or the like, the microneedle may have to be configured such that, for example, needle-shaped portions are formed to have projecting portions provided with hole portions and the body fluid can flow in from the needle-shaped portions. Unfortunately, such needle-shaped portions themselves are fragile, and it is therefore conceivable to increase the strength of the entire microneedle structure by using a base material having a certain strength, thereby suppressing breakage of the needle-shaped portions. Considering the flow paths or the like for the body fluid flowed in from the needle-shaped portions, it is desirable to ensure a wide range of selection for the types of the base material. However, there is a problem in that, while the base material and the needle-shaped portions are bonded, the bonding means has to be one that does not cause troubles in the base material when various types of base materials are used.

The present invention has been made in view of such actual circumstances, and an object of the present invention is to provide a microneedle structure that can reduce the influence of the base material being bonded to the needle-shaped portions while bonding the base material and the needle-shaped portions and that has a high degree of freedom in selecting the base material. Another object of the present invention is to provide a method for producing such a microneedle structure.

### Means for Solving the Problems

To achieve the above objects, first, the present invention provides a microneedle structure comprising a needle-shaped portion on one surface side of a base material, the base material having fluid permeability in its thickness direction, the needle-shaped portion being composed of a composition that contains a low-melting-point resin having a melting point of 150° C. or lower, the needle-shaped portion having a surface and an interior that are formed with hole portions (Invention 1).

In the above invention (Invention 1), the needle-shaped portion is composed of a composition that contains a low-melting-point resin having a melting point of 150° C. or lower, so that when the needle-shaped portion is formed, high-temperature heating is not necessary, and good workability can be obtained at low cost. Moreover, even when the resin is bonded in a molten state to the base material, the base material is not softened, deformed, or burned, and it is possible to reduce the influence of the base material being bonded to the needle-shaped portions and to increase the degree of freedom in selecting the base material.

In the above invention (Invention 1), the needle-shaped portion may be preferably formed with a porous structure (Invention 2).

In the above invention (Invention 1, 2), the low-melting-point resin may be preferably a water-insoluble resin (Invention 3).

In the above invention (Invention 1 to 3), the low-melting-point resin may be preferably a biodegradable resin (Invention 4).

In the above invention (Invention 4), the biodegradable resin may preferably have a monomer acid dissociation constant of 4 or more (Invention 5).

In the above invention (Invention 1 to 5), the low-melting-point resin may be preferably polycaprolactone or a copolymer of caprolactone and another monomer (Invention 6).

In the above invention (Invention 1 to 6), the needle-shaped portion and the base material are directly bonded (Invention 7).

In the above invention (Invention 1 to 7), the base material may be preferably a porous base material (Invention 8).

In the above invention (Invention 8), the porous base material may preferably contain a water-insoluble material (Invention 9).

In the above invention (Invention 9), the water-insoluble material may be preferably a low-melting-point resin having a melting point of 150° C. or lower (Invention 10).

To achieve the above objects, second, the present invention provides a method for producing a microneedle structure comprising: a needle-shaped portion having an interior formed with a hole portion; and a base material having one surface side on which the needle-shaped portion is formed, the method comprising a bonding step of heating a composition containing a low-melting-point resin having a melting point of 150° C. or lower to bond the heated low-melting-point resin and the base material (Invention 11).

In the above invention (Invention 11), the low-melting-point resin having a melting point of 150° C. or lower is heated, and the heated low-melting-point resin and the base material are bonded, so that high-temperature heating is not necessary, and good workability can be obtained at low cost. Moreover, even when the resin is bonded in a molten state to the base material, the base material is not softened, deformed, or burned, and it is possible to reduce the influence of the base material being bonded to the needle-shaped portions and to increase the degree of freedom in selecting the base material.

To achieve the above objects, third, the present invention provides a method for producing a microneedle structure comprising: a needle-shaped portion having an interior formed with a hole portion; and a base material having one surface side on which the needle-shaped portion is formed, the method comprising a formation step of heating a composition containing a low-melting-point resin having a melting point of 150° C. or lower to form a projecting portion on the base material by the composition (Invention 12).

In the above invention (Invention 12), the composition containing the low-melting-point resin having a melting point of 150° C. or lower is heated, and the projecting portion is formed on the base material by the composition, so that high-temperature heating is not necessary, and good workability can be obtained at low cost. Moreover, even when the resin is heated in a state of being bonded to the base material, the resin is not heated at a high temperature; therefore, the base material is not softened, deformed, or burned, and it is possible to reduce the influence of the base material being bonded to the needle-shaped portions and to increase the degree of freedom in selecting the base material.

In the above invention (Invention 11, 12), the low-melting-point resin may be preferably insoluble in water, the composition may preferably contain the water-insoluble low-melting-point resin and a water-soluble material, and the method may preferably comprise a removal step of, after the formation step, removing with water the water-soluble material of the projecting portion formed of the composition to form a hole portion in the projecting portion (Invention 13).

In the above invention (Invention 13), the water-soluble material may preferably have a melting point of 150° C. or lower (Invention 14).

In the above invention (Invention 11 to 14), the method may preferably comprise a filling step of applying the composition containing the low-melting-point resin to a mold having a recessed portion and heating the composition to a melting point of the low-melting-point resin or higher to fill the recessed portion with the composition (Invention 15).

### BRIEF DESCRIPTION OF DRAWINGS

FIG. 1 is a schematic partial cross-sectional diagram of a microneedle structure of the present invention.

FIG. 2 is a cross-sectional diagram of a test patch using the microneedle structure of the present invention.

FIGS. 3A-3G are a set of explanatory diagrams illustrating the procedure of a method for producing a microneedle structure according to the first embodiment.

FIGS. 4A-4C are a set of explanatory diagrams illustrating the procedure of a method for producing a microneedle structure according to the second embodiment.

FIGS. 5A-5E are a set of explanatory diagrams illustrating the procedure of a method for producing a microneedle structure according to the second embodiment.

### EMBODIMENTS FOR CARRYING OUT THE INVENTION

Hereinafter, embodiments of the present invention will be described.

### First Embodiment

### <Microneedle Structure>

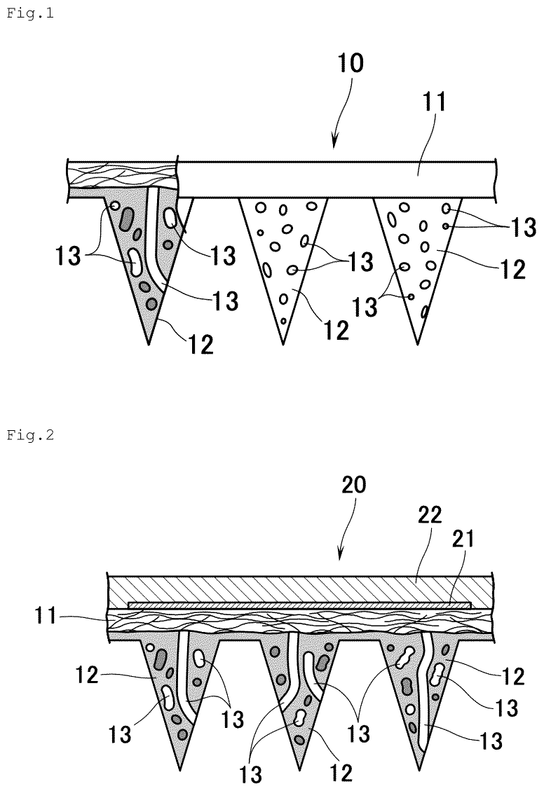

FIG. 1 illustrates a microneedle structure 10 according to an embodiment of the present invention. The microneedle structure 10 includes a plurality of needle-shaped portions 12 that are spaced apart from each other at predetermined intervals on one surface side of a base material 11. The needle-shaped portions 12 are each formed with a plurality of hole portions 13. The microneedle structure 10 can be used as a test patch that absorbs a body fluid with the base material 11 from inside the skin through the hole portions 13 of the needle-shaped portions 12 and performs a test using the obtained body fluid or can also be used as a drug administration patch that administers a drug from the base material 11 into the body through the skin via the hole portions 13 of the needle-shaped portions 12. In the present invention, the body fluid refers to blood, lymph, interstitial fluid, etc.

### (1) Needle-Shaped Portions

The shape, size, formation pitch, and number of formation of the needle-shaped portions 12 can be appropriately selected depending on the intended use of the microneedle. Examples of the shape of the needle-shaped portions 12 include columnar, prismatic, conical, and pyramidal shapes. In the present embodiment, the shape of the needle-shaped portions 12 is pyramidal. The maximum diameter or maximum cross-sectional dimension of the needle-shaped portions 12 may be, for example, 25-1000 μm. The tip diameter or cross-sectional dimension of tips may be 1-100 μm. The height of the needle-shaped portions 12 may be, for example, 50-2000 μm. The needle-shaped portions 12 may be arranged in a plurality of rows in one direction of the base material 11, and each row may be provided with a plurality of needle-shaped portions 12 to form a matrix.

The needle-shaped portions 12 are composed of a low-melting-point resin having a melting point of 150° C. or lower. Materials for the low-melting-point resin are solid at a room temperature and may preferably have a melting point of 150° C. or lower, particularly preferably 40-130° C., and most preferably 45-100° C. When the low-melting-point resin is solid at a room temperature, the needle-shaped portions 12 can maintain their shapes. When the melting point is 150° C. or lower, high-temperature heating is not necessary, and good workability can be obtained at low cost. Moreover, even when the resin is bonded in a molten state to the base material or the resin is heated in a state in which the resin and the base material are bonded, the base material is not softened, deformed, or burned, and the degree of freedom in selecting the base material is high. When the melting point is 130° C. or lower, for example, even if a non-woven fabric or the like whose material is a synthetic fiber or the like having a low heat resistance temperature is used as the base material 11, deterioration of the base material 11 due to softening or the like of the synthetic fiber can be prevented. When the melting point is 100° C. or lower, it is easy to suppress rapid evaporation of a solvent while heating a liquid composition to a temperature equal to or higher than the temperature of a water-insoluble resin in the vibration step, which will be described later.

Such a low-melting-point resin may be preferably a water-insoluble low-melting-point resin. By being water-insoluble, the resin is not dissolved with body fluids when applied to a living body, and it is possible to maintain the shape of the microneedle structure 10 for a desired application time. Moreover, projecting portions can be readily formed with fine hole portions 13, as will be described later. In the present embodiment, the needle-shaped portions 12 are composed of a first water-insoluble material that contains a water-insoluble low-melting-point resin.

Examples of water-insoluble low-melting-point resins other than biodegradable resins, which will be described later, include: polyolefin-based resins such as polyethylene and a-olefin copolymers; olefin copolymer-based resins such as ethylene-vinyl acetate copolymer resins; polyurethane-based elastomers; and acrylic copolymer-based resins such as ethylene-ethyl acrylate copolymers.

The water-insoluble low-melting-point resin may be preferably a low-melting-point biodegradable resin. By being a biodegradable resin, it is possible to reduce the influence on a living body. Preferred examples of such biodegradable resins for use include aliphatic polyesters and derivatives thereof, homopolymers of at least one monomer selected from the group consisting of glycolic acid, lactic acid, and caprolactone, and copolymers composed of two or more monomers. In addition, polybutylene succinate (melting point: 84-115° C.), aliphatic-aromatic copolyester (melting point: 110-120° C.), etc. can also be used as the low-melting-point biodegradable resin. Specific examples of the polybutylene succinate for use include BioPBS provided by Mitsubishi Chemical Corporation, and specific examples of the aliphatic-aromatic copolyester for use include Ecoflex manufactured by BASF.

The low-melting-point biodegradable resin may be preferably a resin whose monomer acid dissociation constant is 4 or more. When the monomer acid dissociation constant is 4 or more, it is possible to reduce the influence on a living body upon application of the microneedle structure of the present invention to the living body. When the monomer is a cyclic ester, the monomer acid dissociation constant as referred to herein is the acid dissociation constant of the hydroxycarboxylic acid resulting from ring-opening of the cyclic ester. The monomer acid dissociation constant may be preferably 4.0 or larger and further preferably 4.5 or larger. From another aspect, the monomer acid dissociation constant may be preferably 25 or less and further preferably 15 or less. Examples of monomers constituting such a biodegradable resin and having an acid dissociation constant of 4 or more include caprolactone. The constituent units of monomers having an acid dissociation constant of 4 or more from which the low-melting-point biodegradable resin is derived may preferably account for 70 mass % or more, more preferably 80 mass % or more, and further preferably 90 mass % or more in the entire constituent units.

More preferred examples of the low-melting-point resin include polycaprolactone or a copolymer of caprolactone and another polymer, which a is water-insoluble and biodegradable resin and whose monomer acid dissociation constant is 4 or more. The molecular weight of the water-insoluble resin is usually 5,000-300,000 and may be preferably 7,000-200,000 and more preferably 8,000-150,000.

In the present embodiment, the needle-shaped portions 12 are described as being composed of a low-melting-point resin, but the needle-shaped portions 12 may contain a resin other than the low-melting-point resin. In this case, the ratio of the low-melting-point resin to the total mass of the resin components contained in the needle-shaped portions 12 may be preferably 50 mass % or more, more preferably 65 mass % or more, and further preferably 80 mass % or more from the viewpoint of efficiently obtaining the effect that the resin can be processed at low temperatures. The needle-shaped portions 12 may further contain a high-melting-point resin having a melting point exceeding 150° C. to such an extent that does not impede the effect that the resin can be processed at low temperatures. Examples of such high-melting-point resins include biodegradable resins such as polyglycolic acid (melting point: 218° C.), polylactic acid (melting point: 170° C.), and polyhydroxybutyric acid (melting point: 175° C.).

Each of the needle-shaped portions 12 has a surface and an interior that are formed with the hole portions 13. The hole portions 13 may be formed in any way, but as in the present embodiment, the hole portions 13 may be preferably formed with porous structures. When each needle-shaped portion 12 is formed so that at least a part thereof has a porous structure, body fluids or medical fluids can pass through the hole portions 13 of the porous structure, so this may be preferred because nano-order flow paths are not necessary to be mechanically formed. Moreover, body fluids or medicinal fluids can flow through all the flow paths of the portion formed with the porous structure in each needle-shaped portion 12, and the amount of flow can therefore be increased as compared with when a simple single communicating hole is formed. Furthermore, in such a case where each needle-shaped portion 12 is formed so that at least a part thereof has a porous structure, when the porous structure is not covered partially or entirely on the side surfaces of a needle-shaped portion, the hole portions 13 are also opened on the side surfaces of that needle-shaped portion 12. In this case, the amount of flow of the fluid can be increased as compared with when only the tip portion of a needle-shaped portion 12 is opened. The hole portions 13 may be formed by removing a first water-soluble material in a removal step, which will be described later, to form voids, and body fluids or medical fluids can pass through the hole portions 13. It is also possible to form porous structures at the same time as forming the needle-shaped portions 12 by using a foam material or the like, or to form porous structures by sintering a particulate composition containing a low-melting-point resin. As illustrated in the cross section, the hole portions 13 may be formed by removing the first water-soluble material to form a plurality of voids that communicate with each other. Some of the hole portions 13 may extend to the base material 11 side. The size of openings of the hole portions 13 may be determined by the application such as a test patch using the microneedle structure 10, but from the viewpoint of facilitating the passage of fluids, the size of the openings may be preferably 0.1-50.0 μm, more preferably 0.5-25.0 μm, and further preferably 1.0-10.0 μm.

### (2) Base Material

The base material 11 has fluid permeability in its thickness direction. Examples of the base material 11 include a porous base material in which a plurality of voids communicate with each other to form fine base material hole portions that penetrate from one surface (the surface provided with the needle-shaped portions 12) to the back surface (the surface opposite to the surface provided with the needle-shaped portions 12) side. In the present invention, a low-melting-point resin is used as the resin forming the needle-shaped portions 12, so various base materials can be selected as the base material 11 depending on the application.

The base material 11 may be plate-like, but a sheet-like one may be preferred because conformability to the skin is high. It may be preferred to use, as the base material 11, a base material composed of a fibrous substance that is easy to handle. Here, the fibrous substance in the present invention means fibers such as natural fibers and chemical fibers. Examples of base materials composed of fibrous substances include nonwoven fabrics, woven fabrics, knitted fabrics, and papers.

In addition to porous base materials, resin films, metal foils, etc. can also be used as the base material 11, and resin films may be preferred from the viewpoint of flexibility and the like. In the present invention, a low-melting-point resin is used as the resin forming the needle-shaped portions 12, so usable ones include polybutylene terephthalate, polyethylene terephthalate, polyethylene, polypropylene, ethylene-vinyl acetate copolymer, vinyl chloride, acrylic resin, polyurethane, and polylactic acid. The use of a film containing such a resin allows the base material 11 to be readily obtained with high flexibility. When using a resin film, it may be preferred to provide through-holes in the resin film so that the fluid can pass through the front and back. The shape of the through-holes is not particularly limited, but a structure in which a plurality of through-holes having a small diameter are provided may be preferred from the viewpoint of ensuring a sufficient amount of flow while causing the capillary action. The diameter of the through-holes may be, for example, 2 mm or less, preferably 0.05-1 mm, and more preferably 0.1-0.8 mm. The method of forming the through-holes is not particularly limited, and the through-holes can be formed, for example, by punching or laser perforation.

The base material 11 may have a structure in which a plurality of layers are laminated. For example, the base material 11 may be a laminate of a first layer of non-woven fabric and a second layer of paper. In this case, any of the first layer and the second layer may be used as the lamination surface with the needle-shaped portions 12. Three or more layers may be laminated depending on the application. A laminate base material may be obtained by laminating a resin film provided with through-holes and porous base material such as nonwoven fabric.

The base material hole portions of the base material 11 may communicate with the hole portions 13 of the needle-shaped portions 12 to form communication holes. The shape of the base material hole portions may be determined by the material of the base material 11. The base material 11 may preferably have a porosity due to the base material hole portions of 1-70%, more preferably 5-50%, and particularly preferably 10-30%. When the porosity falls within this range, the base material 11 can sufficiently absorb the body fluid absorbed by the needle-shaped portions 12.

The needle-shaped portions 12 may be directly bonded to one surface side of the base material 11 as will be described later. For example, when the base material 11 and the needle-shaped portions 12 are bonded by an adhesive layer or the like, gaps may be formed between the base material 11 and the needle-shaped portions 12, and there is a concern that fluid may leak out or the adhesive layer may hinder the passage of fluid between the base material 11 and the needle-shaped portions 12, but when the base material 11 and the needle-shaped portions 12 are directly bonded, their flow paths can be readily connected. In the present embodiment, the first water-insoluble material constituting the needle-shaped portions 12 is a low-melting-point resin, and high-temperature heating is thereby unnecessary; therefore, even when the base material 11 and the needle-shaped portions 12 are bonded, good workability can be obtained at low cost, and there is no risk of softening, deformation, burning, or the like of the base material 11, so the degree of freedom in selecting the base material 11 is high. Specifically, it is possible to suppress curling of the base material due to heat when using paper as the base material, deterioration of the base material caused by softening of fiber due to heat when using nonwoven fabric composed of a resin material having a low softening point, such as polyester non-woven fabric, etc. Moreover, the first water-insoluble material is present even on a portion of one surface of the base material 11 where the needle-shaped portions 12 are not formed, and is in a state of bonding to the base material 11. The first water-insoluble material can thereby serve as a base for individual needle-shaped portions 12 on the whole of the one surface side of the base material 11, and a base portion having hole portions are formed as for the individual needle-shaped portions 12. In the present embodiment, this base portion is composed of the same material as described for the needle-shaped portions 12 or is formed by the same step for the needle-shaped portions 12, and the needle-shaped portions 12 and the base material 11 can therefore obtain good bonding properties via the base portion, which may be preferred. Furthermore, the first water-insoluble material is present even on the portion where the needle-shaped portions 12 are not formed, and is in a state of bonding to the base material 11, and the strength of the microneedle structure 10 as a whole can thereby be further improved. In addition, the area where the needle-shaped portions 12 are bonded to the base material 11 increases, and the bonding properties between the needle-shaped portions 12 and the base material 11 can thereby be improved. From the viewpoint that the base material 11 can have fluid permeability, the base material 11 may preferably maintain the base material hole portions while containing a second water-insoluble material (a water-insoluble material), which will be described later in detail.

### <Test Patch>

The microneedle structure 10 may be preferably used for a test patch 20 that absorbs a body fluid from inside the skin via the needle-shaped portions 12 and performs a test using the obtained body fluid. As illustrated in FIG. 2, the test patch 20 may have the microneedle structure 10 and may further have an analysis sheet 21 and a tape 22 on the back surface side of the base material 11 of the microneedle structure 10. The microneedle structure 10 can also be used as a drug administration patch that administers a drug from the base material 11 into the body through the skin via the needle-shaped portions 12. In this case, the drug administration patch may be configured such that a physiologically active substance-containing sheet is provided on the back surface side of the base material 11 of the microneedle structure 10 and the physiologically active substance can be administered from the physiologically active substance-containing sheet into the body through the skin via the base material 11 and the needle-shaped portions 12.

The analysis sheet 21 may be for analyzing and testing body fluids such as subcutaneous blood and interstitial fluid, and may be installed on the back surface side of the base material 11. When the needle-shaped portions 12 are pierced into the skin of the subject, the body fluid flows through the hole portions 13 of the needle-shaped portions 12, passes through the base material hole portions while being absorbed by the base material 11, and reaches the analysis sheet 21. The analysis sheet 21 for use can be appropriately selected in accordance with the desired test contents and can be formed by incorporating a component as an analysis means into a base material such as paper. Examples of such an analysis sheet 21 include a glucose measurement paper that changes color in accordance with the glucose concentration in the body fluid. When the glucose measurement paper is used as the analysis sheet 21, the test patch 20 for blood glucose level measurement can be obtained, in which the analysis sheet 21 absorbs the interstitial fluid sampled by the microneedle structure 10 and changes color, and the blood glucose level is measured over time based on the degree of color change.

The tape 22 may be composed of a material having biosafety and may be preferably composed of a material having flexibility, stretch properties, and even shrink properties in consideration of the conformability to the skin to which the tape 22 is attached, but is not limited to such a material. Preferred materials for the tape 22 include a stretchable woven fabric, and conventionally known ones can be used.

### <Method for Producing Microneedle Structure>

### (Filling Step)

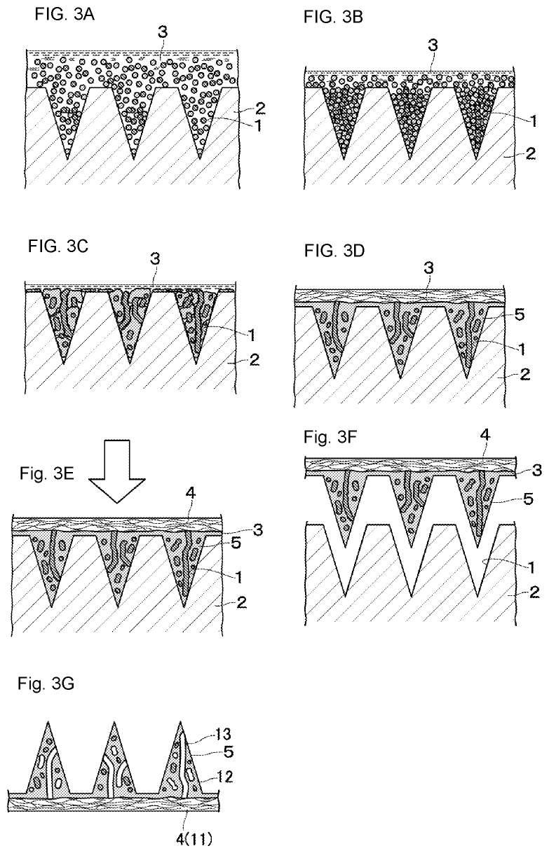

FIGS. 3A-3G illustrates a method for producing the microneedle structure 10 according to an embodiment of the present invention. In the present embodiment, as illustrated in FIG. 3A[[(a)]], a mold 2 having a plurality of recessed portions 1 may be filled with a liquid composition 3 (filling step). The recessed portions 1 may be filled with the liquid composition 3.

The material of the mold 2 is not particularly limited, but the mold 2 may be preferably formed, for example, of a silicone compound or the like that facilitates the creation of an accurate mold and allows the solidified liquid composition 3 to be easily released. In the present embodiment, the mold 2 may be composed of polydimethylsiloxane. The mold 2 may be provided with a wall portion (not illustrated) at the peripheral portion, and the liquid composition 3 poured into the recessed portions 1 within the wall portion can be stored in the mold 2. The recessed portions 1 provided in the mold 2 are for forming the needle-shaped portions 12 illustrated in FIG. 1 and are configured to be able to form the needle-shaped portions 12 having a desired shape. In the mold 2, a plurality of recessed portions 1 are provided at predetermined positions in a plurality of rows at intervals.

The liquid composition 3 contains the aforementioned first water-insoluble material (schematically illustrated as light gray circles in FIG. 3A[[(a))]], a first water-soluble material that is soluble in water (schematically illustrated as dark gray circles in FIG. 3A [[(a))]], and a solvent. For descriptive purposes, the figure illustrates a state in which each material is dispersed in the solvent in a form of particles. In the liquid composition 3, at least one of the first water-insoluble material and the first water-soluble material may be dissolved in the solvent. From the viewpoint of facilitating the formation of the needle-shaped portions 12 with porous structures, at least the first water-insoluble material may be preferably dissolved in the solvent. The viscosity of the liquid composition 3 may be preferably 0.1-1000 mPas, more preferably 0.5-100 mPas, and particularly preferably 1.0-10 mPas. When the viscosity falls within this range, the liquid composition 3 can be poured into the mold 2 with good workability, and the filling ability of the composition into the recessed portions 1 in the filling step is also good; therefore, the desired needle-shaped portions 12 can be formed.

A water-soluble material having a melting point higher than room temperature may be preferred as the first water-soluble material. The water-soluble material may be organic or inorganic, and examples thereof include sodium chloride, potassium chloride, salt cake, sodium carbonate, potassium nitrate, alum, sugar, and water-soluble resin. Among these, the water-soluble resin may be preferred. The water-soluble resin may be preferably a water-soluble thermoplastic resin, and the water-soluble thermoplastic resin may be more preferably a biodegradable resin in consideration of the influence on the human body. Such biodegradable resins include at least one selected from the group consisting of polyalkylene glycols such as polyethylene glycol and polypropylene glycol, polyvinyl alcohol, collagen, and a mixture thereof, and polyethylene glycol may be particularly preferred. The molecular weight of polyethylene glycol may be, for example, preferably 200-4,000, 000, more preferably 600-500,000, and particularly preferably 1,000-100,000.

The water-soluble resin may be preferably a water-soluble resin having a melting point of 150° C. or lower and the melting point of the water-soluble resin may be more preferably 30-130° C. and further preferably 35-100° C. When the melting point is 150° C. or lower, high-temperature heating is not necessary, the base material 11 is not damaged in bonding to the base material 11, and the degree of freedom in selecting the base material 11 is high. When the melting point is 130° C. or lower, even if a non-woven fabric or the like whose material is a synthetic fiber or the like is used as the base material 11, softening or the like due to heating of the synthetic fiber can be prevented. When the melting point is 100° C. or lower, it is easy to suppress rapid evaporation of the solvent while heating the liquid composition to a temperature equal to or higher than the temperature of the first water-soluble material in the vibration step, which will be described later. Examples of such first water-soluble materials include polyethylene glycol and polyvinylpyrrolidone. In the heating step, which will be described later, the difference between the melting point of the first water-insoluble material and the melting point of the first water-soluble material may be preferably 40° C. or smaller and more preferably 30° C. or smaller so that both the first water-insoluble material and the first water-soluble material can be readily melted at the same heating temperature.

The first water-insoluble material and the first water-soluble material may be preferably mixed at a mass ratio of 9:1-1:9, more preferably 8:2-2:8, and particularly preferably 7:3-3:7. When the liquid composition 3 is configured in this ratio, the needle-shaped portions 12 having a desired porosity can be formed, and the needle-shaped portions 12 can readily achieve both the fluid permeability and the strength.

In the present embodiment, the liquid composition 3 may contain a solvent in order to be liquid while containing each material. The solvent may be water or an organic solvent, but when the first water-insoluble material is to be dissolved, the liquid composition 3 may preferably contain an organic solvent. It may be sufficient that the organic solvent can dissolve or disperse the aforementioned first water-insoluble material and first water-soluble material. Examples of such an organic solvent for use include: aliphatic hydrocarbons such as hexane, heptane, and cyclohexane; aromatic hydrocarbons such as toluene and xylene; halogenated hydrocarbons such as methylene chloride and ethylene chloride; alcohols such as methanol, ethanol, propanol, butanol, and 1-methoxy-2-propanol; ketones such as acetone, methyl ethyl ketone, 2-pentanone, isophorone, and cyclohexanone; esters such as ethyl acetate and butyl cellosolve-based solvents such as ethyl acetate; and cellosolve.

The mass-based total content of the first water-insoluble material and the second water-insoluble material in all the components of the liquid composition 3 may be preferably 40% or less, more preferably 35% or less, and particularly preferably 30% or less. When the compositions are contained in this range with respect to the liquid composition 3, the liquid composition 3 can be formed with a desired viscosity that facilitates the production of the needle-shaped portions 12 of the microneedle structure 10, and as a result, it is possible to form the needle-shaped portions 12 in a desired shape.

In the present embodiment, the liquid composition 3 has been described as containing the first water-soluble material and the first water-insoluble material, but the present invention is not limited to this, provided that the liquid composition 3 contains a low-melting-point resin. For example, the liquid composition 3 may contain only the first water-insoluble material (low-melting-point resin). In addition, the liquid composition 3 may contain materials other than the first water-soluble material and the low-melting-point resin as non-volatile solids. For example, in order to further increase the strength of the needle-shaped portions, the water-insoluble material may contain a water-insoluble resin other than the low-melting-point resin, or a component other than the resin, such as silica filler. In this case, the content of the low-melting-point resin in the entire water-insoluble components may be preferably 60 mass % or more, more preferably 75 mass % or more, and further preferably 90 mass % or more.

In the present embodiment, when using the liquid composition 3 in which each material is dispersed in the solvent, the liquid composition 3 may further contain a dispersant.

### (Vibration Step)

Then, it may be preferred to perform a vibration step in which the mold 2 is placed in an ultrasonic cleaning apparatus and subject to ultrasonic vibration. The means for applying vibration is not limited to the ultrasonic cleaning apparatus, provided that it can apply fine vibration to the mold 2. By performing such a vibration step, filling of the recessed portions 1 with the liquid composition 3 is promoted as illustrated in FIG. 3B[[(b)]], and the recessed portions 1 are each filled to every corner with the first water-insoluble material and the first water-soluble material. By this filling, the needle-shaped portions 12 can be formed without defects due to air bubbles and having high transferability sufficiently in accordance with the shape of the recessed portions 1, and the strength of the needle-shaped portions 12 can be improved.

Heating may be performed at the same time as performing the ultrasonic treatment as the vibration step. In this case, it may be preferred to perform heating at a temperature or higher that can promote evaporation/drying of the solvent (e.g., 45° C. or higher) and it may particularly preferred to perform heating at a melting point or higher of the first water-insoluble material (low-melting-point resin) contained in the liquid composition 3. By performing the heating at this temperature, the surface solidification of the liquid composition 3 can be suppressed to promote the evaporation/drying of the solvent, and the filling of the recessed portions 1 with the first water-insoluble material and the first water-soluble material in the liquid composition 3 can be promoted. For example, when the low-melting-point resin is polycaprolactone having a melting point of 60° C., by heating the liquid composition 3 at 60° C. or higher, the evaporation/drying of the solvent can be more promoted, and the filling of the recessed portions 1 with the first water-insoluble material and the first water-soluble material can be further promoted. Similarly, in the vibration step, it may also be preferred to perform heating at a melting point or higher of the first water-soluble material contained in the liquid composition 3.

The frequency in the vibration step may be preferably 10-200 kHz, more preferably 20-150 kHz, and particularly preferably 30-80 kHz. In the vibration step, the time for performing the ultrasonic treatment may be preferably 0.5-10 minutes and more preferably 2-7 minutes. By vibrating the mold 2 in this range, the filling of the recessed portions 1 with the first water-insoluble material and the first water-soluble material in the liquid composition 3 can be further promoted.

### (Deaeration Step)

It may be preferred to perform a deaeration step after the vibration step. This allows the air included in the recessed portions 1 to be removed, and the evaporation/drying of the solvent can be promoted while further promoting the filling of the recessed portions 1 with the first water-insoluble material and the first water-soluble material. When ethyl acetate is used as the solvent, for example, as in Examples described later, the deaeration step may be preferably carried out at 0.01-0.05 MPa and 20-25° C. By performing the deaeration within this pressure range, the solidification of the liquid composition 3 on the surface can be suppressed to promote the evaporation/drying of the solvent, and the filling of the recessed portions 1 with the first water-insoluble material and the first water-soluble material can be further promoted.

### (Heating Step)

After that, it may be preferred to perform a heating step of heating the mold 2. Through this heating step, as illustrated in FIG. 3C [[(c)]], the evaporation/drying of the solvent can be further promoted, the first water-insoluble material can be heated to start softening/deformation, and the filling of the recessed portions 1 with the first water-insoluble material can be promoted.

From the viewpoint of improving the bonding properties between the first water-insoluble material (low-melting-point resin) and the base material 11 while promoting the evaporation of the solvent, the heating temperature may be preferably 40° C. or higher, and the heating may be preferably performed at 180° C. or lower at which the influence on the base material 11 is small. From the viewpoints of improving the bonding properties and reducing the influence of heat on the base material 11, the heating temperature may be more preferably 45-140° C. and further preferably 50-100° C. In relation to the melting point of the first water-insoluble material, it may be preferred to perform heating at a temperature that is not lower than the melting point of the low-melting-point resin and that is not higher than a temperature higher than the melting point of the low-melting-point resin by 30° C., and it may be more preferred to perform heating at a temperature that is not lower than the melting point of the low-melting-point resin and that is not higher than a temperature higher than the melting point of the low-melting-point resin by 20° C. Thus, in the present embodiment, the low-melting-point resin having a low melting point is used, and the temperature of the heating step can thereby be set low. In the present embodiment, it may be preferred to perform heating at a temperature at which the first water-insoluble material and the first water-soluble material, which are low-melting-point resins, can be melted. When it is important to perform heating at a lower temperature, the first water-insoluble material may be heated at a temperature at which it does not melt but starts to soften as described above. However, considering the reduction of the production time, the filling ability of the first water-insoluble material into the recessed portions 1, etc., it may be preferred to perform heating at a temperature that is not lower than the melting point of the low-melting-point resin at which the first water-insoluble material starts to melt as above. When the first water-soluble material is also a resin having a melting point of 150° C. or lower, it may be preferred to heat the liquid composition 3 at a temperature that is not lower than the melting point of the first water-soluble material and that is not higher than a temperature hither than the melting point of the first water-soluble material by 30° C., and the heating temperature may be more preferably a temperature that is not lower than the melting point of the first water-soluble material and that is not higher than a temperature hither than the melting point of the first water-soluble material by 20° C. Even when the first water-soluble material is a resin having a melting point exceeding 150° C., it can be applied in the present embodiment. In the present embodiment, the heating step is performed after the deaeration step, but the heating step may be performed prior to the deaeration step.

When the solvent evaporates/dries in the heating step, the first water-insoluble material and the first water-soluble material contained in the liquid composition 3 stay in the mold 2 in a molten state. That is, as illustrated in FIG. 3D[[(d)]], the recessed portions 1 in the mold 2 are sufficiently filled with the first water-insoluble material and the first water-soluble material. The filling of the recessed portions 1 with the first water-insoluble material and the first water-soluble material is sufficiently performed through the vibration step, the deaeration step, and/or the heating step, so in the present embodiment, the needle-shaped portions 12 can be obtained in a desired shape without defects due to air bubbles and having high transferability sufficiently in accordance with the shape of the recessed portions 1, the strength of the needle-shaped portions 12 can be high, and the bonding properties between the needle-shaped portions 12 and the base material 11 can be satisfactory. The first water-insoluble material and the first water-soluble material overflow from the recessed portions 1 and remain on the surface of the mold 2 formed with the recessed portions 1, but in this state, only the first water-insoluble material and the first water-soluble material are contained and the solvent is substantially not contained. Thus, in the present embodiment, the formation step of forming projecting portions 5 is performed through the filling step and the subsequent vibration step, deaeration step, and heating step.

### (Sheet)

In this state, when a sheet 4 is placed on the mold 2 as illustrated in FIG. 3D [[(d)]], the first water-insoluble material and the first water-soluble material in the recessed portions 1 are melted and therefore bonded to the sheet 4 because the mold 2 is heated in the preceding step. Therefore, high-temperature heating is not necessary, and good workability can be obtained at low cost. Moreover, since the heating temperature in the heating step is low, even if the molten material and the base material 11 come into contact with each other, the base material 11 is not softened/deformed or burned.

Moreover, the first water-insoluble material and the first water-soluble material remain in a molten state also on the bottom surface of the mold 2 formed with the recessed portions 1, and the molten first water-insoluble material and first water-soluble material are thereby bonded to the entire one surface of the sheet 4 to form a base portion. Thus, in the present embodiment, the base portion is composed of the same material as the needle-shaped portions 12 and is formed by the same step, and therefore the needle-shaped portions 12 and the base material 11 can readily obtain good bonding properties via the base portion, which may be preferred. This can improve the bonding properties between the projecting portions 5 (needle-shaped portions 12) and the base material 11 while reinforcing the base material 11 as a whole surface.

The sheet 4 may be obtained such that the aforementioned base material 11 is made to contain a second water-soluble material that is soluble in water and a second water-insoluble material that is insoluble in water. Thus, the sheet 4 contains the second water-insoluble material and the second water-soluble material, and therefore the base material 11 can be prevented from absorbing the molten composition in the recessed portions 1. As a result, even when the microneedle structure 10 includes the base material 11 and is formed using the liquid composition 3, excessive voids are not formed particularly in the elementary portions of the projecting portions 5, and the collapse of the needle-shaped portions 12 can therefore be suppressed. Thus, the needle-shaped portions 12 having a shape suitable for bonding to the base material 11 can be formed, and the microneedle structure 10 in which the needle-shaped portions 12 and the base material 11 are well bonded can be produced.

Furthermore, since the sheet 4 contains not only the second water-soluble material but also the second water-insoluble material, the molten low-melting-point resin in the recessed portions 1 is thermally fusion-bonded to the second water-insoluble material contained in the sheet 4, and the bonding properties between the sheet 4 and the projecting portions 5 can thereby be further improved. In order to further improve such bonding properties, the second water-insoluble material may also be preferably a low-melting-point resin having a melting point of 150° C. or lower and the melting point may be more preferably 40-130° C. and further preferably 45-100° C. As the low-melting-point resin, the same low-melting-point resin as described for the first water-insoluble material can be used. When the second water-insoluble material is a resin, it is easy to impregnate the porous base material 11.

As the second water-soluble material, those described for the first water-soluble material can be used, but the second water-soluble material may be preferably the same as the first water-soluble material. When the second water-soluble material and the first water-soluble material are the same, it may be easy to remove the second water-soluble material and the first water-soluble material in the subsequent removal step, and the desired hole portions 13 of the needle-shaped portions 12 can be formed. The second water-soluble material may also be preferably a resin having a melting point of 150° C. or lower and the melting point of such a resin may be more preferably 30-130° C. and further preferably 35-100° C.

As the second water-insoluble material, those described for the first water-insoluble material can be used, but the second water-insoluble material may be preferably the same as the first water-insoluble material. When the second water-insoluble material and the first water-insoluble material are the same, the thermal fusion bonding between the second water-insoluble material and the first water-insoluble material can be more facilitated, and the bonding properties between the projecting portions 5 and the sheet 4 can be improved.

The second water-soluble material and the second water-insoluble material may be contained in the sheet 4 in any way, but at least the second water-soluble material may be contained so that the sheet 4 does not absorb the first water-insoluble material and first water-soluble material in the recessed portions 1 from the surface side of the base material 11 bonded to the projecting portions 5 (needle-shaped portions 12). That is, the sheet 4 may contain at least the second water-soluble material and may be configured such that the second water-soluble material can block at least some of the base material hole portions of the porous base material 11 thereby to suppress absorption of the first water-insoluble material and the first water-soluble material. For example, a layer that contains the second water-soluble material and the second water-insoluble material may be laminated on the surface of the base material 11 to which the projecting portions 5 are bonded. Preferably, the base material 11 may be immersed in a solution containing the second water-soluble material and the second water-insoluble material thereby to be impregnated with the second water-soluble material and the second water-insoluble material. Additionally or alternatively, an inkjet scheme or the like may be adopted to apply a solution containing the second water-soluble material and the second water-insoluble material to the porous base material 11. The solution impregnated in the porous base material 11 and containing the second water-soluble material and the second water-insoluble material may be dried so that the second water-soluble material and the second water-insoluble material remain in the base material hole portions of the base material 11. This may be a simple means for impregnation and may be preferred.

The solution may further contain a solvent in addition to the second water-soluble material and the second water-insoluble material. The total content concentration of the second water-soluble material and the second water-insoluble material in all the components of the solution may be preferably 1-35%, more preferably 3-30%, and particularly preferably 5-25%. The solution may preferably contain the second water-soluble material and the second water-insoluble material in a mass ratio of 9:1-1:9. When the mass ratio falls within this range, it may be easy to obtain an effect of restoring the base material hole portions of the base material 11 by removing the material in the removal step, which will be described later, and the bonding properties between the base material 11 and the needle-shaped portions 12 can be readily enhanced.

When the base material 11 is immersed in the solution containing the second water-soluble material and the second water-insoluble material, for example, the base material 11 may be immersed in the solution at 10-60° C. for 1-60 minutes, and then the solvent may be volatilized for drying thereby to allow the base material 11 to be impregnated with the second water-soluble material and the second water-insoluble material. In particular, when the base material 11 is composed of a fibrous substance, the base material 11 can readily and sufficiently undergo the absorption of and impregnation with the second water-soluble material and the second water-insoluble material by being immersed in the solution.

In the present embodiment, the sheet 4 is configured to contain the second water-insoluble material, but the present invention is not limited to this. Even if the sheet 4 does not contain the second water-insoluble material, the mold 2 is heated, so the first water-insoluble material and first water-soluble material in the recessed portions 1 are in a molten state, and when the sheet 4 is placed, the projecting portions 5 composed of the first water-insoluble material and first water-soluble material melted in the recessed portions 1 are bonded to the surface of the placed sheet 4.

### (Pressurization Step)

Then, as illustrated in FIG. 3E [[(e)]], a pressurization step may be performed to apply pressure to the sheet 4. The pressurization method is not particularly limited, and a known method can be used. In the pressurization step, the bonding properties can be further improved by performing the heating step at the same time. From the viewpoint of improving the bonding properties of the first water-insoluble material to the base material 11, it may be preferred to perform heating at 40° C. or higher and 180° C. or lower at which the influence on the base material is small, and it may be more preferred to perform heating at 45-140° C. at which the first water-insoluble material and the base material 11 can be bonded well. It may be further preferred to perform heating at 50-100° C. at which the first water-insoluble material starts to melt. Furthermore, in relation to the melting point of the first water-insoluble material, it may be preferred to perform heating at a temperature that is not lower than the melting point of the low-melting-point resin and that is not higher than a temperature higher than the melting point of the low-melting-point resin by 30° C., and it may be more preferred to perform heating at a temperature that is not lower than the melting point of the low-melting-point resin and that is not higher than a temperature higher than the melting point of the low-melting-point resin by 20° C. Thus, in the present embodiment, the use of a low-melting-point resin allows the heating temperature during pressurization to be also set low. Therefore, good workability can be obtained at low cost, and there is no risk that softening/deformation or the like of the base material 11 occurs during the heating step. In the present embodiment, the heating may be performed at a temperature at which the first water-insoluble material and the first water-soluble material, which are low-melting-point resins, can be melted.

After that, the projecting portions 5 in the recessed portions 1 may be solidified by retaining them in a low-temperature state of −10-3° C., and the bonding between the projecting portions 5 and the sheet 4 is completed. Thus, in the present embodiment, the bonding step of bonding the projecting portions 5 and the sheet 4 is performed through the heating step, the subsequent pressurization step, and the solidification of the projecting portions 5. The projecting portions 5 and the sheet 4 may be bonded only by the heating step without performing the pressurization step.

### (Removal Step)

After completion of the bonding step, as illustrated in FIG. 3F[[(f)]], a removal step may be performed to release the solidified projecting portions 5 and sheet 4 bonded together from the mold 2 and then remove the water-soluble materials in the projecting portions 5 and the sheet 4.

The cleaning liquid in this removal step may contain water, and the removal step may be performed, for example, by statically placing the projecting portions 5 and sheet 4 bonded together in the cleaning liquid. By statically placing the projecting portions 5 and sheet 4 bonded together in the cleaning liquid containing water, portions exposed to outside or portions communicating with the portions exposed to outside in the first water-soluble material and the second water-soluble material contained in the projecting portions 5 and the sheet 4 may dissolve and flow into the water and may be removed. The cleaning liquid may be a mixed solvent such as water and alcohol. Through this removal, as illustrated in FIG. 3G[[(g)]], the hole portions 13 may be formed in the projecting portions 5, which may be formed as the needle-shaped portions 12. This allows the microneedle structure 10 to be obtained. In the removal step, the second water-soluble material is removed thereby to at least partially restore the base material hole portions of the base material 11 which are blocked by the second water-soluble material, and the sheet can therefore exhibit good fluid permeability. Moreover, when the sheet 4 contains the second water-soluble material and the second water-insoluble material, the base material 11 can have the restored base material hole portions while containing the second water-insoluble material. In this case, by dissolving a portion in which the first water-soluble material and the second water-soluble material are in contact with each other, the second water-insoluble material remains in the base material 11 while the hole portions 13 extending in the needle-shaped portions 12 to the base material 11 side can be further connected to the base material hole portions of the base material 11. This allows a fluid to readily pass through the interfaces between the needle-shaped portions 12 and the base material 11 in the microneedle structure 10.

### (Modification of Means for Forming Projecting Portions)

In the present embodiment, the first water-insoluble material is used to form the needle-shaped portions 12 in order to readily form the hole portions 13 by removing the first water-soluble material, but the method of creating the hole portions 13 is not particularly limited, provided that the aforementioned low-melting-point resin is used. In any case, by using a low-melting-point resin to form the needle-shaped portions 12, high-temperature heating is not necessary; therefore, good workability can be obtained at low cost, and the base material 11 does not deform/soften, so the degree of freedom in selecting the base material 11 can be increased.

In the present embodiment, the recessed portions 1 are filled with the liquid composition 3 to form the needle-shaped portions 12, but the present invention is not limited to this. For example, the formation step may adopt a scheme that includes: preparing the liquid composition 3 to have a viscosity of 0.1 to 1000 mPas in a state of containing the first water-soluble material and the first water-insoluble material; and dropping the liquid composition 3 with a dispenser or the like on the base material 11 thereby to form the needle-shaped portions 12. Also in this case, the liquid composition 3 can be melted at a low temperature to form the needle-shaped portions 12; therefore, good workability can be obtained at low cost, and even when the base material 11 is indirectly heated, the base material 11 does not deform/soften, so the degree of freedom in selecting the base material 11 can be increased.

### (Method for Producing Test Patch)

Although not illustrated, the test patch 20 can be produced through disposing the analysis sheet 21 at a predetermined position on the back surface side of the base material 11 of the obtained microneedle structure 10 and laminating the tape 22 so as to cover the analysis sheet 21 (installation step). A conventionally known method can be used as the lamination method. For example, the test patch 20 can be produced through placing the analysis sheet 21 on the back surface side of the base material 11 and then laminating the tape 22 in which a pressure sensitive adhesive layer of a rubber-based pressure sensitive adhesive, an acrylic-based pressure sensitive adhesive, a silicone-based pressure sensitive adhesive, or the like is laminated on a tape base material. A drug administration patch can also be produced by a similar method.

### Second Embodiment

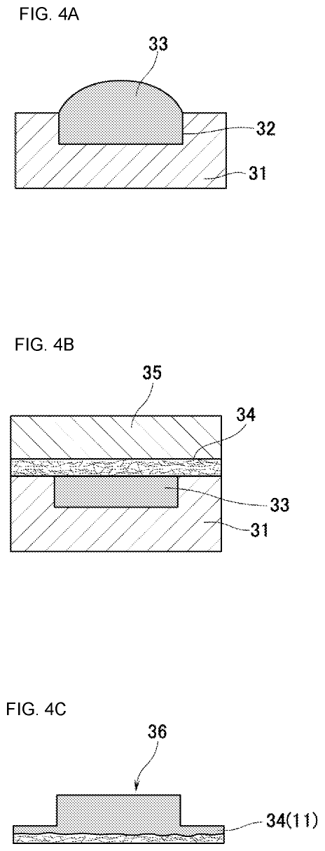

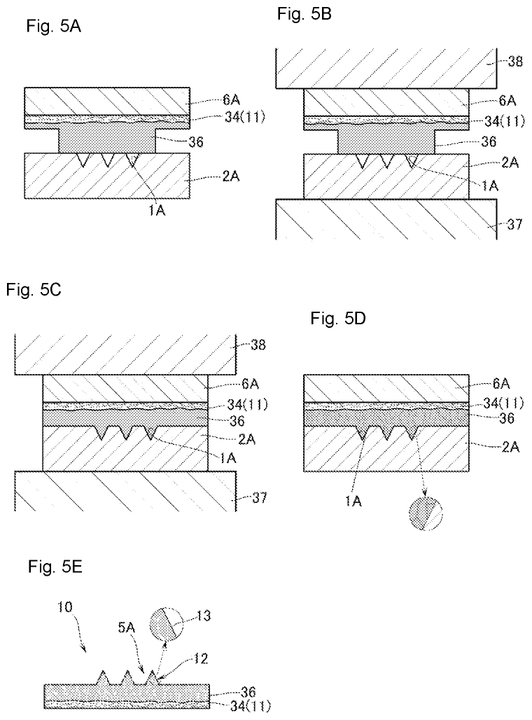

FIGS. 4 and 5 illustrate a method of producing a microneedle structure 10 according to another embodiment of the present invention. The present embodiment is different from the first embodiment in that a solid composition that has a base material 11 and contains a first water-insoluble material and a first water-soluble material is installed on a mold for forming projecting portions and the solid composition is melted to form the projecting portions.

### (Bonding Step)

Preparation of the solid composition with the base material 11 will first be described.

First, the aforementioned first water-insoluble material and the aforementioned first water-soluble material are heated to melt and mixed to prepare a mixture 33. In preparation of the mixture 33, it may be preferred to perform heating at 40° C. or higher and 180° C. or lower at which the influence on the base material is small, more preferably at 55-140° C., and further preferably at 70-120° C. so that the bonding properties of the first water-insoluble material to the base material is improved in the subsequent step and the viscosity is reduced when the resin is melted. Also in the preparation of the mixture 33, the heating temperature can be set low because the low-melting-point resin is used. Accordingly, even when the mixture 33 is bonded to the base material 11 in a molten state in the subsequent step, good workability can be obtained at low cost, and the base material 11 is not softened, deformed, or burned, so the degree of freedom in selecting the base material 11 is high. In the present embodiment, the mixture 33 may be preferably in a molten state. When it is important to perform heating at a lower temperature, the mixture 33 may be softened to such an extent that the mixture 33 is bonded to the base material 11, but considering the reduction of the production time or the like, it may be preferred to perform heating at a temperature that is not lower than the melting point of the low-melting-point resin at which the first water-insoluble material starts to melt as above.

As illustrated in FIG. 4A[[(a)]], the mixture 33 is injected into a recessed portion for solid composition 31 formed in a mold for solid composition 32. When injected, the mixture 33 comes to a state of rising from the surface of the mold for solid composition 31 due to the surface tension. The recessed portion for solid composition 32 may be formed with a shape and a capacity that are able to store a desired amount of the mixture 33.

The material of the mold for solid composition 32 is also not particularly limited, but it may be preferably formed, for example, of a silicone compound or the like, which facilitates the creation of an accurate mold and allows the solidified mixture 33 to be readily released, and may be composed of polydimethylsiloxane in the present embodiment.

For the first water-insoluble material and first water-soluble material used in the mixture 33, those described in the first embodiment can be used. These first water-insoluble material and first water-soluble material may each be mixed in a molten state. The mixing ratio of the first water-insoluble material and the first water-soluble material in the mixture 33 may also be the same as in the first embodiment.

In the present embodiment, the mixture 33 contains the first water-insoluble material and the first water-soluble material, but it may be sufficient that at least a low-melting-point resin is contained. In order to increase the strength of the needle-shaped portions 12, the mixture 33 may contain a water-insoluble resin other than the low-melting-point resin, or a component other than the resin, such as silica filler.

Then, as illustrated in FIG. 4B [[(b)]], a sheet 34 including the base material 11 may be placed on the mold for solid composition 32 so as to cover the molten mixture 33, and the molten mixture 33 may be thereby attached to the molten sheet 34. Even when the molten mixture 33 is attached to the sheet 34, the low-melting-point resin is used in the present embodiment; therefore, the heating temperature is low, so good workability can be obtained at low cost, and the base material 11 is not softened/deformed or burned due to the molten mixture 33.

For the sheet 34, those described as the base material 11 in the first embodiment can be used. In the present embodiment, unlike the first embodiment, when the base material 11 is porous, the sheet 34 may preferably not contain the second water-soluble material and the second water-insoluble material because the molten mixture 33 is absorbed in the base material 11.

Then, a cover 35 (polydimethylsiloxane sheet) for the mold for solid composition 32 may be placed on the sheet 34 and pressed from above. Due to the pressing, the molten mixture 33 protruding from the surface of the mold for solid composition 32 due to the surface tension flows outward from the recessed portion for solid composition 31 while attaching to the sheet 34 and spreads to a portion of the surface of the sheet 34 that does not face the recessed portion for solid composition 31 (the surface facing the mixture 33 among both surfaces of the sheet 34). By pressing, the sheet 34 can be installed at a desired position with respect to the mixture 33. In addition, the molten mixture 33 spreads over the sheet 34 by pressing, and the strength of the base material 11 itself can therefore be increased. Moreover, since the mixture 33 is attached to the sheet 34, the mixture 33 is less likely to further permeate the sheet 34, and the permeation of the composition into the base material 11 can be suppressed in the subsequent step, resulting in suppression of the unintended formation of voids in the elementary portions of the needle-shaped portions 12. Furthermore, by pressing, the mixture 33 may be sufficiently bonded to the sheet 34 thereby to allow the base material 11 to contain the material for forming the needle-shaped portions 12, and the bonding properties between the base material 11 and the needle-shaped portions 12 can thus be improved.

The pressure during the pressing may be preferably 0.1-10.0 MPa. Within this range, the bonding properties between the sheet 34 and the mixture 33 is satisfactory. Moreover, from the viewpoint of improving the bonding properties of the mixture 33 to the base material 11 during the pressing, the mixture 33 may be heated under conditions similar to or different from those described above.

After that, the mixture 33 may be retained at −10-3° C. for 1-60 minutes in a state of bonding to the sheet 34 (refrigeration/solidification step), and the molten mixture 33 may thereby be solidified into a solid state, so the solidified mixture 33 may be released from the mold for solid composition 32 together with the sheet 34. Through this operation, a solid composition 36 provided with the base material 11 can be obtained as illustrated in FIG. 4C [[(c)]].

### (Mold)

Then, using the obtained solid composition 36 provided with the base material 11, preparation of the microneedle structure 10 may be performed.

As illustrated in FIG. 5A[[(a)]], the solid composition 36 provided with the base material 11 may be placed on a mold 2A having recessed portions 1A for forming projecting portions. The mold 2A differs from the mold 2 used in the first embodiment in that the mold 2A has no wall, but otherwise the same, and the recessed portions 1A may be formed under the same conditions as those for the recessed portions 1. The solid composition 36 may be placed so as to face the recessed portions 1A of the mold 2A. A cover 6A for the mold 2A may be installed on the back surface side of the sheet 34.

### (Heating/Pressurization Step)

Then, a heating/pressurization step illustrated in FIGS. 5(b) and 5(c) may be performed. The heating/pressurization step includes a preliminary step (FIG. 5B [[(b))]] for starting to melt the solid composition 36 provided with the base material 11 in order to sufficiently fill the recessed portions 1A of the mold 2A with the solid composition 36 and a main step (FIG. 5C[[(c))]] for sufficiently filling the recessed portions 1A with the molten solid composition 36. The heating/pressurization step can be performed, for example, by a heating/pressing machine. The heating/pressurization step of the second embodiment is a step corresponding to the filling step of the first embodiment.

First, in the preliminary step, as illustrated in FIG. 5B[[(b)]], the sheet 34 is placed so that the solid composition 36 faces the recessed portions 1A, and the sheet 34 is interposed between the mold 2A and the cover 6A. In this state, the mold 2A and the cover 6A are placed on a lower stage 37, and an upper stage 38 is installed on the mold 2A and the cover 6.

As for the heating conditions in the preliminary step and the main step, it may be preferred to perform at least heating at 40° C. or higher and 180° C. or lower at which the influence on the base material 11 is small, more preferably at 55-140° C., and further preferably at 70-120° C. In the present embodiment, the heating may be performed at a temperature at which the solid composition 36 can be melted. In order to heat the solid composition 36, the lower stage 37 may be heated or the upper stage 38 may be heated. In the main step, the heating may be maintained after the preliminary step, and the temperature may be changed as appropriate.

In the present embodiment, the low-melting-point resin is used as the material for forming the needle-shaped portions 12, so the heating temperature in the heating/pressurization step can be set to a low temperature at which the influence on the base material 11 is small. It is thereby possible to obtain good workability at low cost, and there is no risk that the base material 11 is softened/deformed or burned. Furthermore, in this state, the mold 2A may be pressed (pressurized) between the upper stage 38 and the lower stage 37. The pressure in this preliminary step may be preferably 0.1-5.0 MPa. The pressure within this range allows the solid composition 36 to be melted in a short time, and the recessed portions 1A and the like can be quickly filled with the molten solid composition 36. Then, the retention for 10 seconds to 10 minutes leads to a state in which the solid composition 36 is melted. The pressurization conditions may be changed between the preliminary step and the main step. For example, in the main step, pressurization can be performed at a higher pressure or for a longer time than in the preliminary step.

After that, as illustrated in FIG. 5D [[(d)]], the mold 2A is released from the lower stage 37, and the molten solid composition 36 may be retained at −10-3° C. for 1-60 minutes to be refrigerated and solidified (refrigeration/solidification step). This allows the projecting portions 5A to be formed, which have high transferability for a shape corresponding to the recessed portions 1A. Thus, in the present embodiment, the formation step of forming the projecting portions 5 may be performed through the bonding step and the subsequent heating/pressurization step.

### (Removal Step)

Finally, the sheet 34 and the projecting portions 5A are separated from the mold 2A to perform a removal step. The removal step is the same as in the first embodiment. This allows the hole portions 13 to be formed in the projecting portions 5A, and the needle-shaped portions 12 may be formed to obtain the microneedle structure 10, as illustrated in FIG. 5E[[(e)]]. In the present embodiment, the solid composition 36 can be melted at a low temperature to form the needle-shaped portions 12; therefore, good workability can be obtained at low cost, and the base material 11 does not deform/soften, so the degree of freedom in selecting the base material 11 can be increased. The test patch 20 can be produced from the microneedle structure 10 thus obtained.

### (Modification)

In the present embodiment, the solid composition 36 has been described as containing the first water-soluble material and the first water-insoluble material, but the present invention is not particularly limited to this, provided that the solid composition 36 contains at least a low-melting-point resin. For example, the formation step may include filling the mold 2 with a particulate low-melting-point resin or the like and sintering the low-melting-point resin at a temperature equal to or higher than the melting point of the low-melting-point resin thereby to obtain a microneedle structure having a porous structure composed of the sintered particles and a large number of voids formed between the particles. Also in this case, when the formation step and the bonding step are performed at the same time, the solid composition 36 containing a low-melting-point resin can suppress the deformation and deterioration of the base material 11. When the solid composition 36 is used as in the present embodiment, the composition does not contain a solvent, so discoloration and deformation of the base material 11 can be suppressed, which may be preferred. Furthermore, in the present embodiment, the order of the bonding step and the heating/pressurization step may be changed, and the bonding step may be performed after the heating/pressurization step. In this case, as in the first embodiment, the sheet 34 may preferably contain the second water-soluble resin in order to suppress absorption of the mixture 33 by the base material 11.

In the present embodiment, the sheet 34 including the base material 11 is placed so as to cover the molten mixture 33, and the molten mixture 33 is thereby attached to the molten sheet 34, but at this stage, the sheet 34 may not be attached to the mixture 33, and after the solid composition 36 is obtained, the sheet 34 including the base material 11 may be bonded to the solid composition 36 without heating. In this case, the sheet 34 may preferably have an adhesive layer for adhering to the solid composition 36. In this case, the sheet 34 is not heated in the bonding step, but fortunately, by using a low-melting-point resin as the material for forming the needle-shaped portions 12, the heating temperature in the formation step can also be set to a low temperature at which the influence on the base material 11 is small, which may improve the workability at low cost. Moreover, there is no risk that the base material 11 is softened/deformed or burned. In the microneedle structure 10, if the obtained needle-shaped portions 12 or the base portion has a porous structure, the bonding area of the needle-shaped portions 12 or the base portion with respect to the base material 11 will be small, which may be disadvantageous for the bonding properties therebetween. Fortunately, however, the base material 11 and the solid composition 36 are subjected to the heating in the formation step thus in a state of being bonded to each other, and it can thereby be possible to improve the bonding properties between the needle-shaped portions 12 or the base portion and the base material 11.

When the base material 11 is provided with an adhesive layer, gaps may be formed between the base material 11 and the needle-shaped portions 12 as described above, and there is a concern that fluid may leak out or the adhesive layer may hinder the passage of fluid between the base material 11 and the needle-shaped portions 12. It may therefore be preferred to provide an adhesive layer so as to surround the region through which fluid is to pass in the base material 11 and provide a non-adhesive layer formed region in the central portion.

Furthermore, the bonding step may be performed after the formation step. In this case, upon the bonding between the projecting portions 5A or the like before the removal step or the needle-shaped portions 12 or the like after the removal step and the base material 11, even when the bonding involves heating, the material is base 11 not deformed/softened, and the workability is good.

In the present embodiment, the case where the base material 11 is porous has been exemplified, but as the sheet 34, the above-described resin film, metal foil, or the like may be used.

The present invention will be described in more detail below with reference to Examples.

### EXAMPLES

### Example 1

A liquid composition having a solid concentration of 20% was prepared by compounding 100 mass parts of polyethylene glycol (molecular weight: 4000, melting point: 40° C.) as the first water-soluble material, 100 mass parts of polycaprolactone (melting point: 60° C., acid dissociation constant of 6-hydroxycaproic acid as a ring-opened monomer: 4.8) as the first water-insoluble material, and 800 mass parts of ethyl acetate as a solvent (organic solvent). The peripheral portion of a mold composed of polydimethylsiloxane was formed with a wall portion surrounding a square space (15 mm square) when viewed from above. To form the elementary portions of needle-shaped portions with a base portion, 0.7 ml of the liquid composition was poured into the space so that a part of the space within the wall portion would be filled with the liquid composition. Recessed portions formed in the mold are as follows.
Shape of recessed portions: square pyramidal shape with square cross section
Length of one side of maximum cross section of recessed portions: 500 μm
Height of recessed portions: 900 μm
Pitch of recessed portions: 1000 μm
Number of recessed portions: 13 columns, 13 rows, total 169
Size of region formed with recessed portions: 15 mm square
Arrangement of recessed portions: square grid pattern

Then, the mold was placed in an ultrasonic cleaning apparatus (ultrasonic cleaner AU-10C/available from Aiwa Medical Industry Co., Ltd.) and subjected to ultrasonic treatment for 1 minute.

Then, as a deaeration step, vacuum drying was performed for 30 minutes under a reduced pressure environment of a temperature of 23° C. and a pressure of 0.05 MPa. After that, heating was performed at 110° C. for 30 minutes under a non-humidity conditioning environment.

On the other hand, a solution having a solid concentration of 10% was prepared by compounding 100 mass parts of polyethylene glycol (the same as the first water-soluble material) as the second water-soluble material, 100 mass parts of polycaprolactone (the same as the first water-insoluble material) as the second water-insoluble material, and 1800 mass parts of ethyl acetate as a solvent (organic solvent). In addition, a filter paper (WHATMAN FILTER PAPER GRADE4/GE Healthcare Life Sciences) as the base material was immersed in the above solution, then taken out, and dried at a condition of 23° C. for 60 minutes to prepare a sheet.

The pressurization step was performed through placing the sheet on the exposed surface of the base portion provided above the projecting portions formed in the recessed portions of the mold being heated and loading a weight (500 g) on the placed sheet while retaining the heating at 110° C. Then, in a state of being loaded with the weight, the sheet, the mold, etc. were retained at a low temperature state of 3° C. for 10 minutes to solidify the projecting portions and the base portion, and the projecting portions and the base portion were bonded to the sheet. The bonded sheet and the solidified projecting portions and base portion were released from the mold and immersed in purified water at 23° C. for 24 hours to dissolve and remove the first water-soluble material and second water-soluble material in the projecting portions, base, and sheet, thus forming the needle-shaped portions and the base portion.

After that, the projecting portions and base portion bonded together from which the first water-soluble material and the second water-soluble material were removed by dissolution were statically placed under an environment of 23° C. and a relative humidity of 50% for 24 hours and dried by evaporation of water to prepare a microneedle structure.

### Example 2

A mixture was prepared through weighing 100 mass parts of the same polyethylene glycol as in Example 1 as the first water-soluble material and 100 mass parts of the same polycaprolactone as in Example 1 as the first water-insoluble material and melting and mixing them by heating and stirring with a stirrer while heating to 100° C. A mold for solid composition was prepared, composed of polydimethylsiloxane and formed with a recessed portion having a depth of 1.5 mm with a circular opening portion having a diameter of 20 mm. The mixture was injected into the mold so as to fill the recessed portion of the mold.

Then, the same filter paper as in Example 1 was placed as a sheet on the mold for solid composition and a mold cover for solid composition (a sheet composed of polydimethylsiloxane) was placed thereon to allow the mixture to attach to the sheet. This state was retained at 3° C. for 5 minutes, and the molten mixture was solidified into a solid form, so the solidified mixture was released from the mold for solid composition together with the sheet to obtain a solid composition with base material.

Then, the heating/pressurization step was performed using a mold prepared under the same conditions for forming the recessed portions as in Example 1 except that no wall portion was provided. The preliminary step was performed through placing the mold on the lower stage of a heating/pressing machine (AH-1T, available from AS ONE CORPORATION), placing the solid composition with base material on the mold so that it would face the recessed portions, overlapping a 30 mm square polydimethylsiloxane sheet on the solid composition, and pressing them at 2 MPa for 3 minutes while heating only the lower stage of the heating/pressing machine at a set heating temperature of 110° C. After that, the main step was performed in the same manner by pressing at 4 MPa for 30 seconds while still heating only the lower stage of the heating/pressing machine at 110° C. Furthermore, they were stored in a refrigerator at 3° C. for 5 minutes to solidify the composition. After that, the sheet was released from the mold and immersed in purified water at 23° C. for 24 hours to dissolve and remove the first water-soluble material to form needle-shaped portions. Thereafter, the sheet provided with the needle-shaped portions was statically placed under an environment of 23° C. and a relative humidity of 50% for 24 hours and dried by evaporation of water to obtain a microneedle structure.

### Comparative Example 1

As Comparative Example, a microneedle structure was prepared in the same manner as in Example 1 except that polylactic acid having a melting point of 170° C. and an acid dissociation constant of lactic acid as a monomer of 3.08 was used as substitute for polycaprolactone and the heating temperature during and before the pressurization step was set to 230° C.

In Examples 1 and 2 and Comparative Example 1, after the projecting portions were formed by cooling the composition and released from the mold, the inside of the projecting portions was observed with an optical microscope (magnification: 50 times and 100 times) before immersion in purified water, and the number of projecting portions remaining on the base material was counted. The transfer ratio was obtained by calculating the ratio of the remaining number to the total number of designed projecting portions. In the microneedle structures obtained in Examples, the transfer ratio was 50% or more and the transferability was thus high, while in the microneedle structure obtained in Comparative Example, the transfer ratio was less than 50% and the transferability was thus low. It can be considered that the transferability was low in Comparative Example because the base material was deformed due to attachment of the molten material when the projecting portions were formed.

### INDUSTRIAL APPLICABILITY

The microneedle structure of the present invention can be used as a test patch, for example, by placing an analysis sheet on the back surface side and laminating it with a tape.

### DESCRIPTION OF REFERENCE NUMERALS

1, 1A Recessed portion
2, 2A Mold
3, 3A Liquid composition
4, 4A Sheet
5, 5A Projecting portion
10 Microneedle structure
11 Base material
12 Needle-shaped portion
13 Hole portion
20 Test patch
21 Analysis sheet
22 Tape
31 Recessed portion for solid composition
32 Mold for solid composition
33 Mixture
34 Sheet
35 Cover
10) 36 Solid composition

1. A microneedle structure comprising a needle-shaped portion on one surface side of a base material, the base material having fluid permeability in its thickness direction, the needle-shaped portion being composed of a composition that contains a low-melting-point resin having a melting point of 150° C. or lower, the needle-shaped portion having a surface and an interior that are formed with hole portions.

2. The microneedle structure according to claim 1, wherein the needle-shaped portion is formed with a porous structure.

3. The microneedle structure according to claim 1, wherein the low-melting-point resin is a water-insoluble resin.

4. The microneedle structure according to claim 1, wherein the low-melting-point resin is a biodegradable resin.

5. The microneedle structure according to claim 4, wherein the biodegradable resin has a monomer acid dissociation constant of 4 or more.

6. The microneedle structure according to claim 1, wherein the low-melting-point resin is polycaprolactone or a copolymer of caprolactone and another monomer.

7. The microneedle structure according to claim 1, wherein the needle-shaped portion and the base material are directly bonded.

8. The microneedle structure according to claim 1, wherein the base material is a porous base material.

9. The microneedle structure according to claim 8, wherein the porous base material contains a water-insoluble material.

10. The microneedle structure according to claim 9, wherein the water-insoluble material is a low-melting-point resin having a melting point of 150° C. or lower.

11. A method for producing a microneedle structure comprising: a needle-shaped portion having an interior formed with a hole portion; and a base material having one surface side on which the needle-shaped portion is formed,
the method comprising a bonding step of heating a composition containing a low-melting-point resin having a melting point of 150° C. or lower to bond the heated low-melting-point resin and the base material.

12. A method for producing a microneedle structure comprising: a needle-shaped portion having an interior formed with a hole portion; and a base material having one surface side on which the needle-shaped portion is formed,
the method comprising a formation step of heating a composition containing a low-melting-point resin having a melting point of 150° C. or lower to form a projecting portion on the base material by the composition.

13. The method for producing a microneedle structure according to claim 12, wherein
the low-melting-point resin is insoluble in water,
the composition contains the water-insoluble low-melting-point resin and a water-soluble material, and
the method comprises a removal step of, after the formation step, removing with water the water-soluble material of the projecting portion formed of the composition to form a hole portion in the projecting portion.

14. The method for producing a microneedle structure according to claim 13, wherein the water-soluble material has a melting point of 150° C. or lower.

15. The method for producing a microneedle structure according to claim 11, comprising
a filling step of applying the composition containing the low-melting-point resin to a mold having a recessed portion and heating the composition to a melting point of the low-melting-point resin or higher to fill the recessed portion with the composition.
